(19) United States
(12) Patent Application Publication
(10) Pub. No.: US 2023/0190181 A1
(43) Pub. Date: Jun. 22, 2023

(54) BONE FRACTURE RECOVERY MANAGEMENT SENSOR AND ANALYTICS

(71) Applicant: Zimmer, Inc., Warsaw, IN (US)

(72) Inventors: Nolan C. Jones, Warsaw, IN (US); Steven Trizyna, Warsaw, IN (US)

(21) Appl. No.: 18/084,062

(22) Filed: Dec. 19, 2022

Related U.S. Application Data
(60) Provisional application No. 63/291,829, filed on Dec. 20, 2021.

(51) Int. Cl. A61B 5/00; A61B 17/80; A61B 17/72

(52) U.S. Cl. CPC A61B 5/4504; A61B 5/6878; A61B 17/80; A61B 17/72; A61B 5/7282; A61B 2562/0261

(57) ABSTRACT

A system for detecting nonunion or malunion fractures can include an implant, a sensor, and a controller. The implant can be securable to a bone of a patient in a location near a fracture of the bone. The sensor can be connectable to the implant and can be configured to produce a sensor signal based on a condition of the implant near the fracture. The controller can be configured to determine a nonunion or malunion of the fracture based on the sensor signal.

### CLAIM OF PRIORITY

This application claims the benefit of U.S. Provisional Patent Application Ser. No. 63/291,829, filed on Dec. 20, 2021, the benefit of priority of which is claimed hereby, and which is incorporated by reference herein in its entirety.

### BACKGROUND

Orthopedic procedures and prostheses are commonly utilized to repair and/or replace damaged bone and tissue in the human body. For example, plates can be used to repair fractured bones where one or more fasteners (such as screws) can be used to secure the plate to the bone. On occasion, fractures supported by plates do not heal properly, which is commonly referred to as a nonunion or malunion fracture. In such cases, further surgical invention is often required to address the complication.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a perspective view of a system.

FIG. 2 illustrates a perspective view of a system.

FIG. 3 illustrates a perspective view of a system.

FIG. 4A illustrates a perspective view of a system.

FIG. 4B illustrates an enlarged perspective view of a system.

FIG. 5 illustrates a schematic view of a method.

FIG. 6 illustrates a block diagram illustrating an example of a machine upon which one or more embodiments may be implemented.

### DETAILED DESCRIPTION

Femoral fractures are one of the most common bone fractures. Of these, periprosthetic and distal femoral fractures can be challenging with where about 20 percent result in a malunion/nonunion rate in distal femoral fractures. Further interventions are often required to address these issues. With current implants, patients are sent home with a therapy protocol and return after weeks or months for follow-up imaging (e.g., X-rays) to check on healing. While this process can detect malunions or nonunions, detection can be delayed from occurrence.

The present disclosure can help to address these issues by allowing information regarding the plate or the bone to be assessed as often as needed or desired by the surgeon to help detect malunions or nonunions as early as possible, allowing physicians to alter care or to intervene earlier, both of which can improve ultimate outcomes. Such detections can be done using a plate system that includes one or more sensors for detecting conditions (e.g., strain) of the plate. These values can be regularly monitored to watch for changes or patterns to detect malunions or nonunions.

The above discussion is intended to provide an overview of subject matter of the present patent application. It is not intended to provide an exclusive or exhaustive explanation of the invention. The description below is included to provide further information about the present patent application.

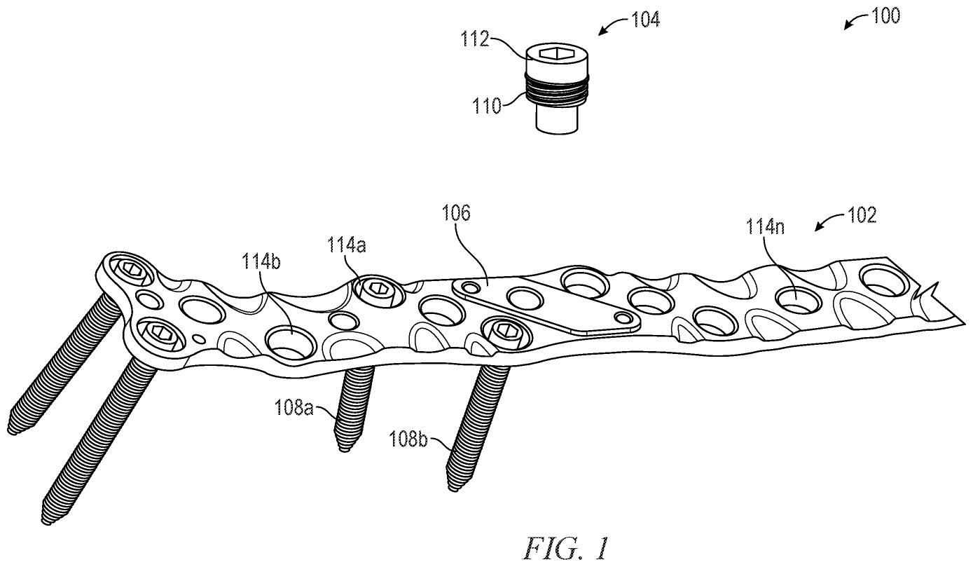

FIG. 1 illustrates a front view of a system 100. The system 100 can include an implant assembly 102 and a sensor 104. The implant assembly 102 can include a bone plate 106 and fasteners 108. The bone plate 106 can define a plurality of bores therein or therethrough.

The sensor 104 can be a sensor configured to produce a signal as a function of a measured force, stress, or strain, such as a load cell, strain load cell, or the like. For example, the sensor 104 can be a linear, shear, or half-bridge strain sensor. The sensor 104 can optionally include one or more Piezoelectric sensors. The sensor 104 cab include a threaded portion 110 and a sensor portion 112 connected to the threaded portion 110. Optionally, the threaded portion 110 can be or include a snap interface or other connection interface for securing the sensor 104 to the plate 106.

The bone plate 106 can be a rigid or semi-rigid and elongate body. The bone plate 106 can be made of materials such as one or more of metals, plastics, foams, elastomers, ceramics, composites, or the like. In some examples, the implant bone plate 106 can be comprised of biocompatible materials such as such as one or more of stainless steels, cobalt-chromium, titanium variations, polyether ether ketone (PEEK), polyether ketone ketone (PEKK) or the like. The fasteners 108 can be nails, screws, or the like for securing the bone plate 106 to a bone.

The fasteners 108 can each be securable to a bone and to the bone plate 106 such as through bores 114a-114n of the bone plate 106. Each of the bores 114 can be configured to receive a fastener 108 or a sensor 104. For example, the bore 114a can receive the fastener 108a therein and the bore 114b can remain open and can optionally receive the sensor 104 therein. The threaded portion 110 can be threadably securable to any of the bores 114a-114n such that the sensor 104 can be placed in the bone plate 106 as desired, as discussed below in further detail.

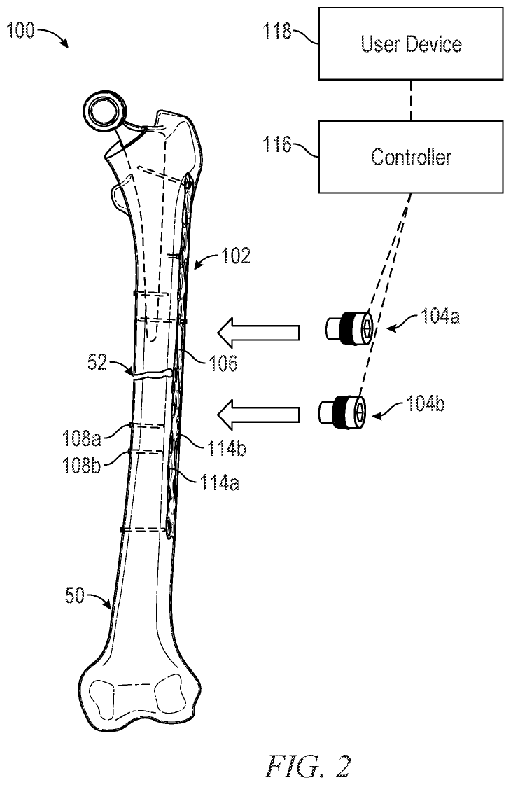

FIG. 2 illustrates a perspective view of the system 100. The system 100 can be similar to the system 100 discussed above with respect to FIG. 1. FIG. 2 shows how the system 100 can be used. FIG. 2 also shows a bone 50 with a fracture 52.

FIG. 2 also shows a controller 116 and a user device 118. The controller 116 can be a programmable controller, such as a single or multi-board computer, a direct digital controller (DDC), a programmable logic controller (PLC), or the like. In other examples the controller 116 can be any computing device, such as a handheld computer, for example, a smart phone, a tablet, a laptop, a desktop computer, or any other computing device including a processor, memory, and communication capabilities. The controller 116 can be a local device configured to communicate with the sensors 104a and 104b before, during, or after implantation in the bone plate 106. The controller 116 can communicate with the sensors 104 through wireless communication, such as Bluetooth, near field contact (NFC), wi-fi, other electromagnetic based communication protocols, or the like. The user device 118 can be or include another controller (optionally similar to the controller 116) and can be one or more of a handheld computer, for example, a smart phone, a tablet, a laptop, a desktop computer, or any other computing device including a processor, memory, and communication capabilities. The user device 118 can be located in the same location as the controller 116 or can be a remote device in remote communication with the controller 116.

In operation, the bone plate 106 of the system 100 can be secured to the bone 50 such as about and spanning the fracture 52 of the bone 50. Fasteners 108 (such as fasteners 108a and 108b) can be secured to the bone 50 to secure the bone plate 106 to the bone 50. Then, sensors 104 can be secured to the bone plate 106. For example, the sensor 104a can be inserted into the bore 114a and the sensor 104b can be inserted into the bore 114b. The sensors 104 can be threadably secured to their respective bores.

The sensor 104a can be positioned on a first side of the fracture 52 and the sensor 104b can be positioned on a second side of the fracture 52. In other examples, more or fewer sensors can be used. In other examples, the sensors 104 can be placed in any bore of the bone plate 106, as desired. The sensors 104 can optionally be connected to the bone plate 106 before placement of the bone plate 106. Optionally, locations of the sensors 104 relative to the bone plate 106 or the bone 50 can be recorded in the controller 116 when placed or can be determined by the controller 116, such as through communication between the sensor 104 and the controller 116.

Following placement and securing of the bone plate 106 to the bone 50, the sensors 104 can communicate with the controller 116, which can optionally communicate with the user device 118. The sensors 104 can transmit a signal to the controller 116 which the controller 116 can use to determine a condition of the bone plate 106 or the bone 50. For example, the sensors 104 can produce a sensor signal indicative of a strain condition of the bone plate 106 or the bone 50 and can transmit the signal to the controller 116. The controller 116 can use the signal to determine whether a malunion or nonunion of the fracture 52 of the bone 50 is present. Optionally, the controller 116 can receive the signal at a time interval (e.g., every second, every hour, or every day) and can determine a strain value at each time interval and store the value. The controller 116 can use the stored values to determine whether a malunion or nonunion of the fracture 52 of the bone 50 is present.

The controller 116 can also compare a new or present strain value to stored strain values to determine whether a malunion or nonunion of the fracture 52 of the bone 50 is present. For example, when a malunion occurs, a strain on the bone plate 106 can be larger than a strain from prior to the malunion occurring. When the increase in strain is detected by the controller 116, the controller 116 can determine that a malunion or nonunion is present in the bone 50.

Also, the controller 116 can compare stored strain values to determine that the fracture 52 is healing correctly. For example, in a normal healing fracture, strain on the bone plate 106 should steadily decrease over time while the fracture heals, such as over 8 weeks, as the plate 106 is loaded less and the bone 50 is loaded more. If the controller 116 determines that the strain in the bone plate 106 does not decrease (or otherwise change) after a period of time (e.g., 4 weeks), the controller 116 can determine that a malunion or nonunion can be present in the bone 50 or can be likely to form. That is, lack of reduction in load or strain on the plate 106 can be an early indication of a malunion or nonunion of the bone 50, which can be detected by the controller 116 and communicate (e.g., through the user device 118) to a physician or surgeon.

Because the sensor 104 can be placed in any bore of the bone plate 106, the sensor 104 can be located (such as by the surgeon) near the fracture 52. A location near the fracture can be a location of the bone plate 106 that will have strain values deviating most over the course of healing of the fracture 52, which can allow the controller 116 to provide more meaningful analysis of the condition of the bone plate 106 and the fracture 52. For example, strain values at proximal and distal ends of the bone plate 106 may not change much between installation, healing, or malunion, but strain on the plate 106 should be highest or most variable near the fracture. By placing the sensor 104 near the fracture 52 the strain detected by the sensor 104 is more likely to deviate over the course of healing or lack thereof, which can help to increase an accuracy of detected malunion or nonunion of the fracture 52.

Further, by using sensors that are securable to the plate 106, as opposed to integrated into the plate 106, cost can be reduced as a custom plate is not required and sensors can be added only when a surgeon deems it is useful. For example, when a patient has a type of fracture more likely to result in a malunion or nonunion, or when the patient is more prone to such problems (e.g., advanced age), the surgeon may incorporate one or more sensors. Also, being able to use one or more sensors also can help the surgeon balance cost and analysis. For example, the surgeon can use two or more sensors for patients with a high likelihood of malunion or nonunion and only one sensor for patients with a lower likelihood.

Also, when using the sensor 104a and the sensor 104b placed on opposing sides of the fracture 52, strain values of the bone plate 106 on either side of a fracture can be determined. By logging and analyzing multiple strain values, the controller 116 can more accurately detect the presence of a malunion or nonunion of the fracture 52. For example, if the current strain values of either the first or second sensor signal are significantly different than the stored values from either or both sensors 104, the controller 116 can determine the presence of a malunion or nonunion. Similarly, during healing, if the strain of either of the signals of the sensor 104a or the sensor 104b does not steadily decrease over time, the controller 116 can detect the presence of a malunion or nonunion.

The controller 116 or the user device 118 can also use historical data or other data for comparison to the current and stored strain values. For example, the user device 118 can have a log of strain values from other patients or of an ideal model. The present or stored values of the sensors 104 can be compared to the log or model to determine the presence of a malunion or nonunion in the bone 50.

Though the above (and below) discussion focus on measuring strain of implants, any of the sensors can measure force (tensile or compressive), stress, acceleration, strain, or the like, of the plate, the implant, the sensor, or other components of the assemblies. For example, the sensor can include one or more of a strain gauge, temperature sensor, accelerometer, gyroscope, load cell, other force sensor, or the like.

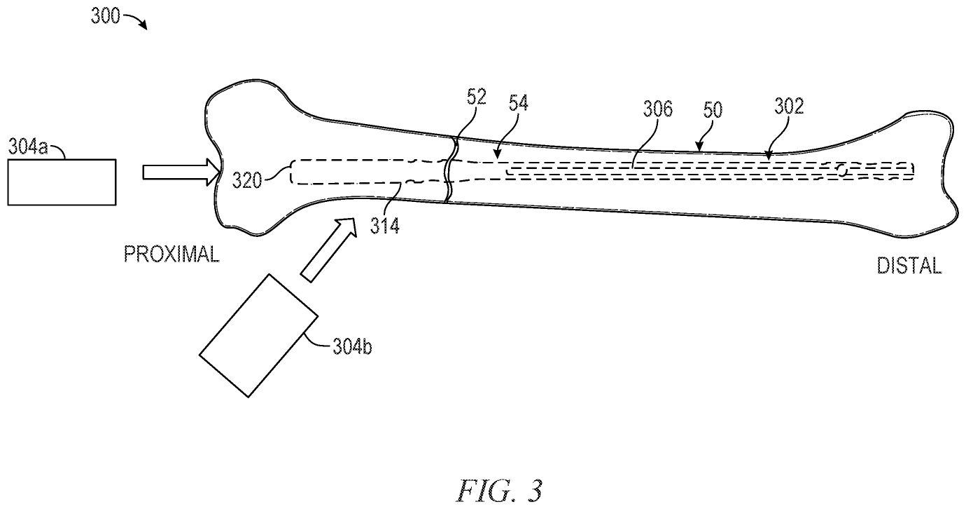

FIG. 3 illustrates a perspective view of a system 300. The system 300 can be similar to the system 100 discussed above; the system 300 can be different in that the system 300 can include an intramedullary implant. Any of the systems discussed above or below can include such an implant. FIG. 3 also shows a bone 50 having a fracture 52 and defining an intramedullary canal 54.

The system 300 can include an implant assembly 302, a sensor 304a, and a sensor 304b (collectively referred to as the sensors 304). The assembly 302 can include an implant 306 and fasteners. The implant 306 be a rigid or semi-rigid and elongate body. The implant 306 can be made of materials such as one or more of metals, plastics, foams, elastomers, ceramics, composites, or the like. In some examples, the implant 306 can be comprised of biocompatible materials such as such as one or more of stainless steels, cobalt-chromium, titanium variations, polyether ether ketone (PEEK), polyether ketone ketone (PEKK) or the like. The fasteners can be nails, screws, or the like for securing the implant 306 to a bone.

The implant 306 can include bores 314 therein, such as in a distal portion of the implant 306. Each of the bores 314 can be configured to receive a sensor therein, such as the sensor 304b. The sensor 304b can be placed in any of the bores 314, as determined by the surgeon, before or after implantation of the implant 306 in the intramedullary canal 54 of the bone 50. For example, the sensor 304b can be placed in a bore 314 that will be near the fracture 52 following implantation of the implant 306 within the intramedullary canal 54 of the bone 50, which can help to provide data, such as strain data, that can be more useful for determination of a malunion or nonunion of the fracture 52.

The implant 306 can also include an elongate bore 320 that can extend along a length of the implant 306. The elongate bore 320 can be configured to receive the sensor 304a therein at any location along the elongate bore 320. The surgeon can optionally place the sensor 304a through a proximal opening in the implant 306 at a location that is estimated or determined to be located near the fracture 52 following implantation of the implant 306 in the intramedullary canal 54 of the bone 50. The sensor 304a or the sensor 304b can be connectable to a controller such as for communicating therewith. The sensors 304 can transmit signals to the controller for the controller to determine strain (or other conditions) of the implant 306 such as for determining the presence of a malunion or nonunion in the bone 50. The controller can use any of the processes discussed above or below.

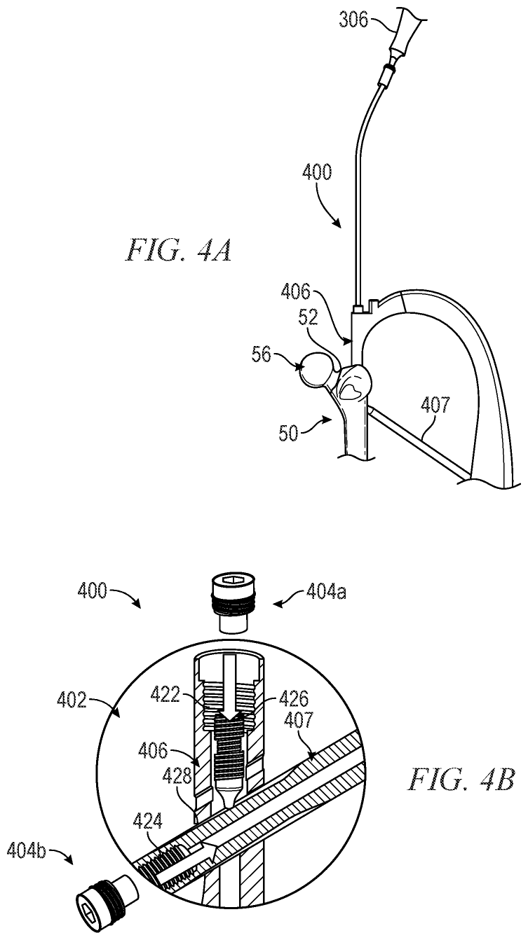

FIG. 4A illustrates a perspective view of a system 400. FIG. 4B illustrates an enlarged perspective view of the system 400. FIGS. 4A and 4B are discussed together below. The system 400 can be similar to the systems discussed above; the system 400 can be different in that the system 400 can include an implant for a neck fracture, such as a femoral neck fracture. Any of the systems discussed above or below can include such an implant. FIG. 4A also shows a bone 50 having a fracture 52 near a head 56 of the bone 50.

The system 400 can include an implant assembly 402, a sensor 404a, and a sensor 404b (collectively referred to as the sensors 404). The assembly 402 can include an implant 406, a lag screw 407, and fasteners. The implant 406 be a rigid or semi-rigid and elongate body, such as a nail or intramedullary implant. The lag screw 407 can be a rigid or semi-rigid and elongate body, such as a lag screw or implant securable to the implant 406 and the head 56 of the bone 50. The implant 406 and the lag screw 407 can be made of materials such as one or more of metals, plastics, foams, elastomers, ceramics, composites, or the like.

The implant 406 can include an elongate bore 422 that can extend along a length of the implant 406. The elongate bore 422 can be configured to receive the sensor 404a therein at any location along the elongate bore 422. The surgeon can optionally place the sensor 404a at a location that is estimated or determined to be located near the fracture 52 following implantation of the implant 406 in the bone 50. The sensor 404a can be threadably secured to the elongate bore 422 near a set screw 426 or can be integrated with the set screw 426. Optionally, a surgeon can use a guide wire or other insertion tool to insert the sensor 404a into the elongate bore 422, such as to a position along a length of the elongate bore 422 near the fracture.

The lag screw 407 can include an elongate bore 424 that can extend along a length of the lag screw 407. The elongate bore 424 can be configured to receive the sensor 404b therein at any location along the elongate bore 424. The surgeon can optionally place the sensor 404b at a location that is estimated or determined to be located near the fracture 52 following implantation of the implant 406 in the bone 50. The sensor 404b can be threadably secured to the lag screw 407. The lag screw 407 can be insertable through a transverse bore 428 of the implant 406 to secure the lag screw 407 to the implant 406 and the head 56 to the bone 50, the implant 406, and the lag screw 407. The set screw 426 can be engageable with the lag screw 407 when the lag screw 407 is located within the transverse bore 428 such as to secure the lag screw 407 within the transverse bore 428. Optionally, a surgeon can use a guide wire or other insertion tool to insert the sensor 404b into the elongate bore 424, such as to a position along a length of the elongate bore 424 near the fracture. By locating the sensors 404 near the fracture, more accurate force or strain data can be gathered by the sensors 404.

The sensor 404a or the sensor 404b can be connectable to a controller such as for communicating therewith. The sensors 404 can transmit signals to the controller for the controller to determine strain (or other conditions) of the implant 406 or 407 such as for determining the presence of a malunion or nonunion in the bone 50. The controller can use any of the processes discussed above or below. Additionally, either of the sensors 404 can be used to detect migration (e.g., any movement) of the set screw 426 or the lag screw 407 with respect to the implant 406.

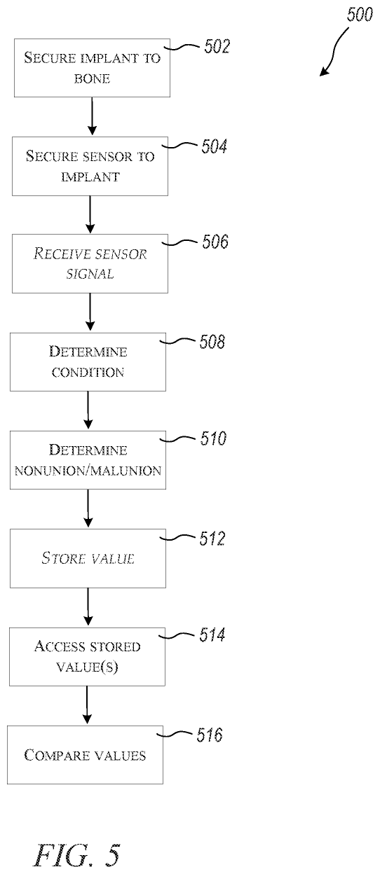

FIG. 5 illustrates a schematic view of the method 500, in accordance with at least one example of this disclosure. The method 500 can be a method of determining nonunion or malunion fractures. More specific examples of the method 500 are discussed below. The steps or operations of the method 500 are illustrated in a particular order for convenience and clarity; many of the discussed operations can be performed in a different sequence or in parallel without materially impacting other operations. The method 500 as discussed includes operations performed by multiple different actors, devices, and/or systems. It is understood that subsets of the operations discussed in the method 500 can be attributable to a single actor, device, or system could be considered a separate standalone process or method.

The method 500 can begin at step 502, where an implant can be secured to the bone. For example, the bone plate 106 can be secured to the bone 50. At step 504, the sensor can be secured to the implant in a location near the fracture. For example, the sensor 104a can be secured to the bone plate 106 near the fracture 52 of the bone 50.

At step 506, a sensor signal from a sensor connected to an implant can be received where the sensor signal can be indicative of a strain applied to the implant near a fracture of the bone. For example, a sensor signal from the sensor 104a connected to the implant 106 can be received where the sensor signal can be indicative of a strain applied to the implant 106 near the fracture 52 of the bone 50.

At step 508, a condition of the implant or bone can be determined. For example, a current strain value of the implant 106 can be determined based on the sensor signal. At step 510, a determination of whether a nonunion or malunion of the fracture of the bone can be made based on the current strain value. For example, it can be determined whether a malunion or nonunion of the bone 50 is present based on the strain value from one or more of the sensor 104.

At step 512, the strain value (or values) can be stored, such as in one or more of the controller 116 or the user device 118. At step 514, one or more of the stored strain values can be accessed, such as by the controller 116 or the user device 118. At step 516, the values can be compared and it can be determined whether a nonunion or malunion of the fracture of the bone exists based on the current value and the plurality of stored values.

In some examples, the current strain value can be compared to the plurality of stored strain values to determine whether a nonunion or malunion of the fracture of the bone exists. In some examples, the plurality of stored strain values can be previously determined strained values. In some examples, the current strain value can be stored with the plurality of stored strain values after determining whether there is a malunion or nonunion of the fracture. In some examples, a location of the strain sensor can be used determine whether a nonunion or malunion of the fracture of the bone exists. In some examples, a second sensor can be secured to the implant in a location on an opposite side of the fracture from the sensor.

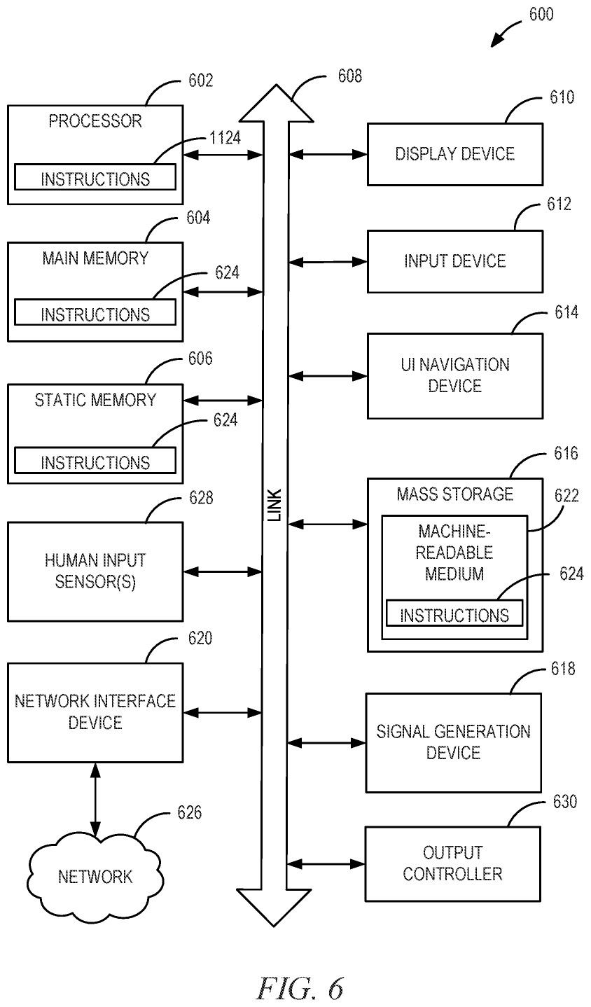

FIG. 6 illustrates a block diagram of an example machine 600 upon which any one or more of the techniques (e.g., methodologies) discussed herein may perform. Examples, as described herein, may include, or may operate by, logic or a number of components, or mechanisms in the machine 600. Circuitry (e.g., processing circuitry) is a collection of circuits implemented in tangible entities of the machine 600 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 600 may include a hardware processor 602 (e.g., a central processing unit (CPU), a graphics processing unit (GPU), a hardware processor core, or any combination thereof), a main memory 604, a static memory (e.g., memory or storage for firmware, microcode, a basic-input-output (BIOS), unified extensible firmware interface (UEFI), etc.) 606, and mass storage 608 (e.g., hard drive, tape drive, flash storage, or other block devices) some or all of which may communicate with each other via an interlink (e.g., bus) 630. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 608, a signal generation device 618 (e.g., a speaker), a network interface device 620, and one or more sensors 616, such as a global positioning system (GPS) sensor, compass, accelerometer, or other sensor. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 602, the main memory 604, the static memory 606, or the mass storage 608 may be, or include, a machine readable medium 622 on which is stored one or more sets of data structures or instructions 624 (e.g., software) embodying or utilized by any one or more of the techniques or functions described herein. The instructions 624 may also reside, completely or at least partially, within any of registers of the processor 602, the main memory 604, the static memory 606, or the mass storage 608 during execution thereof by the machine 600. In an example, one or any combination of the hardware processor 602, the main memory 604, the static memory 606, or the mass storage 608 may constitute the machine readable media 622. While the machine readable medium 622 is illustrated as a single medium, the term “machine readable medium” may include a single medium or multiple media (e.g., a centralized or distributed database, and/or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software. A transmission medium is a machine readable medium.

### NOTES AND EXAMPLES

The following, non-limiting examples, detail certain aspects of the present subject matter to solve the challenges and provide the benefits discussed herein, among others.

Example 1 is a system for detecting nonunion or malunion fractures, the system comprising: an implant securable to a bone of a patient in a location near a fracture of the bone; a sensor connectable to the implant and configured to produce a sensor signal based on a condition of the implant near the fracture; and a controller configured to determine a nonunion or malunion of the fracture based on the sensor signal.

In Example 2, the subject matter of Example 1 includes, wherein the implant is a bone plate securable to an outer surface of the bone, spanning the fracture, the bone plate defining a plurality of bores therein each bore configured to receive a bone fastener therethrough to secure the bone plate to the bone, and wherein the sensor is positionable in any bore of the plurality of bores.

In Example 3, the subject matter of Example 2 includes, a second sensor positionable in any bore of the plurality of bores, the second sensor and configured to produce a second sensor signal based on a second condition of the implant near the fracture.

In Example 4, the subject matter of Example 3 includes, wherein the controller is configured to determine the nonunion or malunion of the fracture based on the sensor signal and the second sensor signal.

In Example 5, the subject matter of Examples 3-4 includes, wherein the sensor is locatable in a first bore of the plurality of bores on a first side of the fracture and the second sensor is locatable in a second bore of the plurality of bores on a second side of the fracture.

In Example 6, the subject matter of Examples 2-5 includes, wherein the sensor is configured to produce a signal based on a strain applied to the bone plate.

In Example 7, the subject matter of Examples 1-6 includes, wherein the implant is an intramedullary nail securable within an intramedullary canal of the bone spanning the fracture, the intramedullary nail defining a plurality of bores therein, and wherein the sensor is positionable in any bore of the plurality of bores to locate the sensor near the fracture.

In Example 8, the subject matter of Example 7 includes, a second sensor positionable in any bore of the plurality of bores, the second sensor configured to produce a second sensor signal based on a second condition of the implant near the fracture.

In Example 9, the subject matter of Example 8 includes, wherein the controller is configured to determine the nonunion or malunion of the fracture based on the sensor signal and the second sensor signal.

In Example 10, the subject matter of Examples 7-9 includes, wherein the intramedullary nail defines an elongate bore extending axially through at least a portion of the intramedullary nail, the sensor locatable at any axial position within the elongate bore to locate the sensor near the fracture.

In Example 11, the subject matter of Examples 1-10 includes, wherein the implant is a lag screw securable to a head of the bone spanning the fracture, the lag screw defining an elongate bore therein, and wherein the sensor is positionable in the elongate bore at any axial position within the elongate bore to locate the sensor near the fracture.

In Example 12, the subject matter of Example 11 includes, an intramedullary nail securable within an intramedullary canal of the bone and engageable with the lag screw, the intramedullary nail defining a nail bore extending axially therein.

In Example 13, the subject matter of Example 12 includes, a second sensor locatable at any axial position within the nail bore to locate the second sensor near the fracture, the second sensor configured to produce a second sensor signal based on a second condition of the implant near the fracture.

In Example 14, the subject matter of Example 13 includes, wherein the controller is configured to determine the nonunion or malunion of the fracture based on the sensor signal and the second sensor signal.

Example 15 is a non-transitory machine-readable medium including instructions, for determining nonunion or malunion fractures of a bone, which when executed by a machine, cause the machine to: receive a sensor signal from a sensor connected to an implant, the sensor signal indicative of a strain applied to the implant near a fracture of the bone; determine a current strain value of the implant based on the sensor signal; access a plurality of stored strain values; and determine whether a nonunion or malunion of the fracture of the bone exists based on the current strain value.

In Example 16, the subject matter of Example 15 includes, the instructions to further cause the machine to: access a plurality of stored strain values; and determine whether a nonunion or malunion of the fracture of the bone exists based on the current value and the plurality of stored values.

In Example 17, the subject matter of Example 16 includes, the instructions to further cause the machine to: compare the current strain value to the plurality of stored strain values to determine whether a nonunion or malunion of the fracture of the bone exists.

In Example 18, the subject matter of Example 17 includes, wherein the plurality of stored strain values are previously determined strained values.

In Example 19, the subject matter of Example 18 includes, wherein the current strain value is stored with the plurality of stored strain values after determining whether there is a malunion or nonunion of the fracture.

In Example 20, the subject matter of Examples 17-19 includes, the instructions to further cause the machine to: determine a location of the strain sensor to determine whether a nonunion or malunion of the fracture of the bone exists.

Example 21 is a method of determining nonunion or malunion fractures of a bone, the method comprising: receiving a sensor signal from a sensor connected to an implant, the sensor signal indicative of a strain applied to the implant near a fracture of the bone; determining a current strain value of the implant based on the sensor signal; accessing a plurality of stored strain values; and determining whether a nonunion or malunion of the fracture of the bone exists based on the current strain value.

In Example 22, the subject matter of Example 21 includes, accessing a plurality of stored strain values; and determining whether a nonunion or malunion of the fracture of the bone exists based on the current value and the plurality of stored values.

In Example 23, the subject matter of Example 22 includes, comparing the current strain value to the plurality of stored strain values to determine whether a nonunion or malunion of the fracture of the bone exists.

In Example 24, the subject matter of Example 23 includes, wherein the plurality of stored strain values are previously determined strained values.

In Example 25, the subject matter of Example 24 includes, wherein the current strain value is stored with the plurality of stored strain values after determining whether there is a malunion or nonunion of the fracture.

In Example 26, the subject matter of Examples 23-25 includes, determining a location of the strain sensor to determine whether a nonunion or malunion of the fracture of the bone exists.

In Example 27, the subject matter of Examples 21-26 includes, securing the implant to a bone; securing the sensor to the implant in a location near the fracture.

In Example 28, the subject matter of Example 27 includes, securing a second sensor to the implant in a location on an opposite side of the fracture from the sensor.

Example 29 is at least one machine-readable medium including instructions that, when executed by processing circuitry, cause the processing circuitry to perform operations to implement of any of Examples 1-28.

Example 30 is an apparatus comprising means to implement of any of Examples 1-28.

Example 31 is a system to implement of any of Examples 1-28.

Example 32 is a method to implement of any of Examples 1-28.

In Example 33, the devices or method of any one or any combination of Examples 1-32 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A system for detecting nonunion or malunion fractures, the system comprising:
an implant securable to a bone of a patient in a location near a fracture of the bone;
a sensor connectable to the implant and configured to produce a sensor signal based on a condition of the implant near the fracture; and
a controller configured to determine a nonunion or malunion of the fracture based on the sensor signal.

2. The system of claim 1, wherein the implant is a bone plate securable to an outer surface of the bone, spanning the fracture, the bone plate defining a plurality of bores therein each bore configured to receive a bone fastener therethrough to secure the bone plate to the bone, and wherein the sensor is positionable in any bore of the plurality of bores.

3. The system of claim 2, further comprising:
a second sensor positionable in any bore of the plurality of bores, the second sensor and configured to produce a second sensor signal based on a second condition of the implant near the fracture.

4. The system of claim 3, wherein the controller is configured to determine the nonunion or malunion of the fracture based on the sensor signal and the second sensor signal.

5. The system of claim 3, wherein the sensor is locatable in a first bore of the plurality of bores on a first side of the fracture and the second sensor is locatable in a second bore of the plurality of bores on a second side of the fracture.

6. The system of claim 2, wherein the sensor is configured to produce a signal based on a strain applied to the bone plate.

7. The system of claim 1, wherein the implant is an intramedullary nail securable within an intramedullary canal of the bone spanning the fracture, the intramedullary nail defining a plurality of bores therein, and wherein the sensor is positionable in any bore of the plurality of bores to locate the sensor near the fracture.

8. The system of claim 7, further comprising:
a second sensor positionable in any bore of the plurality of bores, the second sensor configured to produce a second sensor signal based on a second condition of the implant near the fracture.

9. The system of claim 8, wherein the controller is configured to determine the nonunion or malunion of the fracture based on the sensor signal and the second sensor signal.

10. The system of claim 7, wherein the intramedullary nail defines an elongate bore extending axially through at least a portion of the intramedullary nail, the sensor locatable at any axial position within the elongate bore to locate the sensor near the fracture.

11. The system of claim 1, wherein the implant is a lag screw securable to a head of the bone spanning the fracture, the lag screw defining an elongate bore therein, and wherein the sensor is positionable in the elongate bore at any axial position within the elongate bore to locate the sensor near the fracture.

12. The system of claim 11, further comprising:
an intramedullary nail securable within an intramedullary canal of the bone and engageable with the lag screw, the intramedullary nail defining a nail bore extending axially therein.

13. The system of claim 12, further comprising:
a second sensor locatable at any axial position within the nail bore to locate the second sensor near the fracture, the second sensor configured to produce a second sensor signal based on a second condition of the implant near the fracture.

14. The system of claim 13, wherein the controller is configured to determine the nonunion or malunion of the fracture based on the sensor signal and the second sensor signal.

15. A non-transitory machine-readable medium including instructions, for determining nonunion or malunion fractures of a bone, which when executed by a machine, cause the machine to:
receive a sensor signal from a sensor connected to an implant, the sensor signal indicative of a strain applied to the implant near a fracture of the bone;
determine a current strain value of the implant based on the sensor signal;
access a plurality of stored strain values; and
determine whether a nonunion or malunion of the fracture of the bone exists based on the current strain value.

16. The non-transitory machine-readable medium of claim 15, the instructions to further cause the machine to:
access a plurality of stored strain values; and
determine whether a nonunion or malunion of the fracture of the bone exists based on the current value and the plurality of stored values.

17. The non-transitory machine-readable medium of claim 16, the instructions to further cause the machine to:
compare the current strain value to the plurality of stored strain values to determine whether a nonunion or malunion of the fracture of the bone exists.

18. The non-transitory machine-readable medium of claim 17, wherein the plurality of stored strain values are previously determined strained values.

19. The non-transitory machine-readable medium of claim 18, wherein the current strain value is stored with the plurality of stored strain values after determining whether there is a malunion or nonunion of the fracture.

20. The non-transitory machine-readable medium of claim 17, the instructions to further cause the machine to:
determine a location of the strain sensor to determine whether a nonunion or malunion of the fracture of the bone exists.
